(19) United States
(12) Patent Application Publication
(10) Pub. No.: US 2026/0256954 A1
(43) Pub. Date: Sep. 3, 2026

(54) COMBINATION THERAPY FOR TREATING CANCER

(71) Applicant: ISM BIOSCIENCES LLC, Miami, FL (US)

(72) Inventors: Max Temnik, Miami, FL (US); Victor Gurin, Richmond, VA (US); Sergey Gurin, Richmond, VA (US)

(73) Assignee: ISM BIOSCIENCES LLC, Miami, FL (US)

(21) Appl. No.: 19/068,709

(22) Filed: Mar. 3, 2025

(51) Int. Cl. A61K 51/04; A61K 31/337; A61K 31/4164; A61K 31/704

(52) U.S. Cl. CPC A61K 51/0406; A61K 31/337; A61K 31/4164; A61K 31/704; A61K 2121/00

(57) ABSTRACT

A method of treating cancer or cancer metastasis comprising administering to a patient in need thereof a therapeutically effective amount of one or more anticancer agent selected from the group consisting of chemotherapy, an immunotherapy, a kinase inhibitor, a monoclonal antibody, and an antibody-drug conjugate, and at least one isotope selective modulator inclusive of at least one isotope selected from the group consisting of ⁶³Cu, ⁶⁴Zn, ⁶⁶Zn, and ⁸⁵Rb, wherein said isotope is enriched to exceed 99% atomic mass.

### FIELD

This disclosure relates to a method of treating cancer by combining at least one anticancer drug selected from the group consisting of a chemotherapeutic agent, an immunotherapeutic agent, a kinase inhibitor, a monoclonal antibody, and an antibody-drug conjugate with at least one isotope selective modulator inclusive of at least one enriched isotope selected from the group consisting of ⁶³Cu, ⁶⁴Zn, ⁶⁶Zn, and ⁸⁵Rb.

### BACKGROUND

Several classes of cancer treatment pharmaceuticals are known and widely used. These classes have evolved significantly over the years, offering more targeted and effective treatment options for various types of cancer. These treatments can be effective but typically cause a wide range of adverse effects affecting healthy cells. These side effects can significantly impact patients' physical and emotional well-being, potentially leading to complications, treatment delays, or even discontinuation of treatment that often leads to death. Developing therapies that could improve the efficacy of known treatments while helping the body manage the side effects of cancer treatments is needed.

### SUMMARY

In one aspect, a method of treating cancer is provided comprising administering to a patient in need thereof a therapeutically effective amount of one or more antic-cancer agents selected from the group consisting of a chemotherapeutic agent, an immunotherapeutic agent, a kinase inhibitor, a monoclonal antibody, and an antibody-drug conjugate, and at least one isotope selective modulator inclusive of at least one isotope selected from the group consisting of ⁶³Cu, ⁶⁴Zn, ⁶⁶Zn, and ⁸⁵Rb, wherein said isotope is enriched to exceed 99% atomic mass.

In another aspect, a method of treating cancer metastases is provided comprising administering to a patient in need thereof a therapeutically effective amount of one or more anti-cancer agents selected from the group consisting of a chemotherapeutic agent, an immunotherapeutic agent, a kinase inhibitor, a monoclonal antibody, and an antibody-drug conjugate, and at least one isotope selective modulator inclusive of at least one isotope selected from ⁶³Cu, ⁶⁴Zn, ⁶⁶Zn, or ⁸⁵Rb, wherein said isotope is enriched to exceed 99% atomic mass.

### BRIEF DESCRIPTION OF DRAWINGS

FIG. 1 illustrates the experimental data of combining an isotope selective modulator (⁶⁴Znₑ-Aspartate) with a chemotherapy agent (Paclitaxel).

FIG. 2A, FIG. 2B and FIG. 2C illustrate the experimental data in support of FIG. 1.

FIG. 3 illustrates the experimental data of combining a chemotherapy agent (Paclitaxel) with an isotope selective modulator (⁶⁴Znₑ-Aspartate).

FIG. 4A, FIG. 4B, and FIG. 4C illustrate the experimental data of combining an isotope selective modulator (⁶⁴Znₑ-Aspartate) with a chemotherapy agent (Dacarbazine).

FIG. 5 illustrates the experimental data of combining an isotope selective modulator (⁶⁴Znₑ-Aspartate) with a chemotherapy agent (Dacarbazine).

FIG. 6 illustrates the experimental data of combining an isotope selective modulator (⁶⁴Znₑ-Aspartate) with a chemotherapy agent (Doxorubicin).

### DETAILED DESCRIPTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. All ranges disclosed herein are also to be considered to include the end points of the range, unless expressly stated otherwise. For example, a range of “between 5 and 10” or “5 to 10” or “5-10” should be considered to include the end points 5 and 10.

The feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein shall have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the term “chemical element” refers to any substance that cannot be decomposed into simpler substances by chemical processes. Elements are the fundamental materials of which all matter is composed.

The term “isotope” refers to a variant of an atom of a chemical element that differs by the number of neutrons in its nucleus and has a different atomic mass. According to the proton-neutron model developed by D.I. Ivanenko and W. Heisenberg (1932), atoms of all chemical elements consist of three types of elementary particles: positively charged protons, negatively charged electrons, and neutrons that have no charge. The number of protons p in the nucleus determines the atomic number Z of the chemical element in Mendeleev's periodic table. The proton and the neutron, which have a common name-nucleons—have almost identical weight. The mass of the neutron (1.00866 amu) is somewhat greater than the proton mass (1.00727 amu). The electron mass is much smaller than that of the nucleons (for example, the proton-to-electron mass ratio is 1836.13). Therefore, the mass of the atom is concentrated in its nucleus. Hence, the mass number of the atom A is connected with the atomic number by a simple relation A=p+n=Z+n, where n is the number of neutrons in the nucleus of an atom. The number of protons in the nucleus of an atom uniquely determines the position of an element in the periodic table of the elements. Furthermore, the number of protons determines the number of electrons present in a neutral atom thus determining the chemical properties of this atom. However, atoms with the same atomic number Z (and hence the number of protons p) may have different neutron numbers n. Thus, atoms with different atomic mass numbers may occupy the same position on the periodic table. Chemical elements having the same atomic number but a different atomic mass are known as isotopes.

Stable isotopes are a known matter. Although radioactive isotopes have been used in medicine for diagnostics and radiation therapies, the use of enriched stable isotopes has been limited to the applications in diagnostics, clinical pharmacology, and metabolic studies, all of which have used stable isotopes in their naturally abundant ratios. In diagnostics, stable isotopes have been used as tracers for dynamically assessing in vivo metabolism. In research, stable isotopes have been used in evaluating bioavailability and the release profile of drug products and drug delivery systems. In the assessment of drug pharmacology, stable isotopes have been used for the determination of a drug's pharmacokinetic profile, mechanism of action, and potential toxicity or adverse effects. In precision medicine, stable isotopes have recently started to be used monitoring drug treatment effects and conducting clinical toxicology studies. In nutrition, stable isotopes have been used to assess body composition, energy expenditure, protein turnover, food safety and metabolic profiles. However, all of these prior art techniques and technologies have used stable isotopes in their naturally occurring isotopic ratios.

The term “natural abundance” of an isotope refers to the fraction of the total amount of the corresponding element that the isotope represents, on a mole-fraction basis (that is, not, for example, on a mass basis). For example, ⁶⁴Zn has a natural abundance fraction of 48.63%, that would mean that 48.63% of Zn atoms on earth are the isotope ⁶⁴Zn. When a composition is “enriched” for a certain isotope, the abundance of the isotope in the composition is greater than the isotope's natural abundance. For the preceding ⁶⁴Zn example, a composition in which ⁶⁴Zn constitutes more than 48.63% of the total Zn in the composition, on a mole-fraction basis, would be “enriched” for ⁶⁴Zn. Throughout this disclosure, a subscript “e” following a light isotope chemical symbol or element name indicates that the designated element is enriched for that isotope. For example, ⁶⁴Zn refers to the light isotope zinc-64, whereas ⁶⁴Znₑ refers to zinc that is enriched for zinc-64. Thus, “⁶⁴Znₑ aspartate,” for example, refers to zinc aspartate in which the zinc is enriched for zinc-64.

The term “relative isotopic fraction” refers to a measure of the proportion of one isotope of a chemical element relative to another isotope of the sample of the same element, typically expressed in comparison to a standard reference. For example, the isotopic fraction of ⁶⁴Zn atoms of naturally occurring zinc is 48.6%.

The term “enriched” refers to the process of increasing the proportional fraction of a specific isotope within a mixture of isotopes. The enrichment process exploits the differences in physical and/or chemical properties between isotopes of the same element to separate and concentrate one isotope from the others. The term “isotopically modified” refers to the process of enriching an identified isotope. Hence, terms “enrichment” and “isotopic modification” essentially refer to the same concept. Isotope enrichment is also a known process of isotopic modification that refers to increasing the fraction of a specific isotope in a mixture of isotopes in a chemical element. An enrichment of light isotopes automatically results in a depletion of heavy isotopes of the same chemical element, and vice versa.

The term “therapeutic efficacy” refers to the ability of a medical intervention, such as a pharmaceutical composition or treatment method, to produce a beneficial effect under ideal and controlled circumstances. It is a critical concept in pharmacology and medicine, used to evaluate the potential of new treatments.

A “reduction” of a symptom or symptoms refers to decreasing the severity or frequency of the symptom(s), or elimination of the symptom(s) ascertainable by one skilled in the art using known techniques.

The term “effective amount” is an amount sufficient to accomplish a stated purpose (e.g. achieve the effect for which it is administered, treat a disease, reduce enzyme activity, increase enzyme activity, reduce one or more symptoms of a disease or condition). For any compound described herein, the therapeutically effective amount can be initially determined from clinical trials disclosure, as applicable. Target concentrations will be those concentrations of co-administered active compounds that are capable of achieving the methods described herein using the practices known in the art. An example of an “effective amount” is an amount sufficient to contribute to the treatment, prevention, or reduction of a symptom or symptoms of a disease, which could also be referred to as a “therapeutically effective amount.”

An effective amount can be first estimated either in accordance with cell culture assays or using animal models, typically mice, rats, guinea pigs, rabbits, dogs or pigs. An animal model may be used to determine an appropriate concentration range and route of administration. Such information can then be used to determine appropriate doses and routes of administration for humans. When calculating a human equivalent dose, it is recommended to use a conversion table given in the Guidance for Industry and the Reviewers document (2002, US Food and Drug Administration, Rockville, MD, USA). An exact effective dose will depend on the severity of the disease, patient's general state of health, age, body weight and sex, nutrition, time and frequency of administration, combination(s) of medicines, response sensitivity and tolerance/response to administration and other factors that will be taken into account by a person skilled in the art when determining the dosage and route of administration for a particular patient based on his/her knowledge of the art. Such dose may be determined by conducting routine experiments and at the physician's discretion. Effective doses will also vary depending on the possibility of their combined use with other therapeutic procedures, such as the use of other agents.

A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). “Effective amount,” “prophylactically effective amount,” or “therapeutically effective amount” may refer to an amount of an agent or composition that provides a biological, therapeutic, and/or prophylactic result.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. lymphocytic leukemia or cutaneous melanoma, and severity of the symptoms), type of concurrent treatment, complications and adverse effects from the disease being treated or other patient-related problems. Adjustment and manipulation of established dosages (e.g., frequency and duration) are within the ability of those skilled in the art.

The methods provided by the present disclosure include pharmaceutical compositions wherein the active ingredient (e.g. one or more of a chemotherapy agent, an immunotherapy agent, a kinase inhibitor, a monoclonal antibody, or an antibody-drug conjugate, and one or more isotope selective modulator(s)) is contained in a therapeutically effective amount, i.e., in an amount effective to achieve its intended purpose. The actual amount effective for a particular application will depend, inter alia, on the condition being treated and on patient specific biomarkers. When administered in methods to treat a disease, such compositions will contain an amount of active ingredient effective to achieve the desired result, e.g., modulating the activity of a target molecule (e.g. NF-kB, p53, Bax, etc.), and/or reducing, eliminating, or slowing the progression of disease symptoms or diminishing adverse effects. Determination of a therapeutically effective amount of a compound of the disclosure is within the capabilities of those skilled in the art.

The term “molecule” is known in the art. Briefly, it refers to an electrically neutral group of two or more atoms held together by chemical bonds. Molecules form the smallest identifiable unit of a chemical compound that retains the composition and chemical properties of that compound. Examples include proteins, carbohydrates, lipids, ribonucleic acid, etc.

The term “isotope selective modulator” refers to a pharmaceutical composition that increases or decreases the level or the function of a target molecule. For example and in some embodiments, the isotope selective modulator ⁶⁴Znₑ(Asp) is a modulator of diverse biological function of metallothionein proteins in relation to zinc-mediated cellular processes. ⁶⁴Znₑ(Asp) is an isotope selective modulator of metallothionein based on metallothionein′ cysteine sulfur atoms to coordinate isotopically light zinc. In this example, ⁶⁴Znₑ(Asp) induces expression and activity of metallothionein, which in turn act as zinc chaperones, regulating the isotopically modified zinc ions within cells where isotopic effects of ⁶⁴Znₑ enhance zinc-related effect on cellular functions through several pathways. This may, in turn, further enhance a therapeutic effect rendered by a chemotherapy, an immunotherapy, a kinase inhibitor, a monoclonal antibody, or an antibody-drug conjugate concurrently alleviating a harm rendered to normal, non-malignant cells by a chemotherapy, an immunotherapy, a kinase inhibitor, a monoclonal antibody, or an antibody-drug conjugate therapy.

The term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any suitable route, including parenteral (e.g., intravenous, intramuscular, intra-arteriole, intradermal, subcutaneous, intraperitoneal, intraventricular, and intracranial) and transmucosal (e.g., buccal, sublingual, palatal, gingival, nasal, vaginal, rectal, or transdermal). Other modes of delivery include, but are not limited to, the use of liposomal formulations, intravenous infusion, transdermal patches, etc.

The isotope selective modulators of the present disclosure may be co-administered to the patient. The term “co-administer” means that an isotope selective modulator of the present disclosure is administered at the same time, prior to, or after the administration of one or more of a chemotherapy, an immunotherapy, a kinase inhibitor, a monoclonal antibody, or an antibody-drug conjugate pharmaceutical. Thus, the preparations can also be combined, when desired, with other active substances (e.g. to reduce metabolic degradation, to reduce adverse effects, etc.)

In certain embodiments, co-administration includes administering an isotope selective modulator within 0.25, 0.5, 1, 2, 3, 5, 8, 13, 21, or 24 hours after administering an active agent selected from a chemotherapy, an immunotherapy, a kinase inhibitor, a monoclonal antibody, or an antibody-drug conjugate. Co-administration includes administering two active agents simultaneously, approximately simultaneously (e.g., within about 1, 5, 10, 15, 20, or 30 minutes apart of each other), or sequentially in any order. In some embodiments, simultaneous co-administration can be accomplished by co-formulation, i.e., preparing a single pharmaceutical composition including both active agents. In other embodiments, the active agents can be formulated separately. In the embodiments, the active and/or adjunctive agents may be linked or conjugated to one another. In the embodiments, the compounds described in this disclosure may be combined with surgery.

The isotope selective modulators of the present disclosure may be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The isotope selective modulators of the present disclosure may also be delivered as nanoparticles. The isotope selective modulators of the present disclosure can also be delivered as microspheres for slow release in the body. For example, microspheres can be administered via intratumoral injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). The isotope selective modulators of the present disclosure may be delivered transdermally, by a topical route, formulated as applicator sticks, solutions, suspensions, emulsions, gels, creams, ointments, pastes, jellies, paints, powders, and aerosols. Oral preparations containing the isotope selective modulators of the present disclosure may include tablets, pills, powder, dragees, capsules, liquids, lozenges, cachets, gels, syrups, slurries, suspensions, etc., suitable for ingestion by the patient.

Solid form preparations may include powders, tablets, pills, capsules, cachets, suppositories, and dispersible granules. Liquid form preparations may include solutions, suspensions, and emulsions, for example, water or water/propylene glycol solutions.

The isotope selective modulators of the present disclosure may additionally include components to provide sustained release and/or comfort. Such components include high molecular weight, anionic mucomimetic polymers, gelling polysaccharides and finely divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination, electrocardiogram, echocardiography, radio-imaging, nuclear scan, and/or stress testing, neuropsychiatric exams, and/or a psychiatric evaluation.

The terms “patient”, “individual”, and “subject” are interchangeable terms that refer to a living organism suffering from or prone to a disease or condition that can be treated by administration of the method provided herein. Non-limiting examples include humans, other mammals, bovines, rats, mice, dogs, monkeys, goat, sheep, cows, deer, and other non-mammalian animals. In certain embodiments, a patient is a human subject.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

It should be noted that any embodiment of the invention can optionally exclude one or more embodiment for purposes of claiming the subject matter.

Several classes of pharmaceuticals for cancer treatment are known and widely used. These classes have evolved significantly over the years, offering more targeted and effective treatment options for various types of cancer. These treatments are effective but typically cause a wide range of adverse effects affecting healthy cells. These side effects can significantly impact patients' physical and emotional well-being, potentially leading to complications, treatment delays, or even discontinuation of treatment that often leads to death. Developing therapies that could improve the efficacy of known treatments while helping the body manage the side effects of cancer treatments is essential.

Chemotherapy works by targeting rapidly dividing cells, which is characteristic of cancer cells. It is effective for many types of cancer and is commonly used in cases where cancer has metastasized. Chemotherapy typically shrinks tumors before surgery or eliminates the remaining cancer post-surgery, but it is notorious for its side effects and harmful effect on healthy, rapidly dividing cells like those in the hair, digestive system, and bone marrow.

Immunotherapy works by improving the body's ability to use the immune system for recognizing and fighting cancer cells. This treatment has shown high efficacy in cancers like melanoma, non-small cell lung cancer, and certain types of lymphomas. However, not all cancer patients respond to immunotherapy, and it can lead to immune-related side effects (e.g. inflammation of healthy tissues).

Kinase inhibitors work by blocking the action of enzymes that play critical roles in signaling pathways responsible for cancer cell growth, survival, and division. By blocking these enzymes, kinase inhibitors aim to stop cancer cells from proliferating and spreading. The efficacy of kinase inhibitors varies depending on the specific type of cancer, the targeted mutation, and how well the drug is tolerated by the patient. These side effects of kinase inhibitor treatments are related to the inhibition of normal cellular functions in addition to cancer cell growth and range from mild to severe.

Monoclonal antibody (mAb) therapies have become a cornerstone of modern cancer treatment due to their ability to target specific molecules involved in cancer cell growth, survival, and spread. Unlike traditional therapies, such as chemotherapy or radiation, which affect both cancerous and healthy cells, monoclonal antibodies are designed to selectively bind to cancer cells or tumor marker molecules. Their efficacy depends on several factors, including the cancer type, the target of the antibody, and whether the tumor expresses the specific target. While monoclonal antibody therapies are generally considered to have fewer adverse effects compared to traditional chemotherapy, they are not without risks. One of the most common side effects of monoclonal antibody treatments is an infusion-related reaction (IRR). These reactions can occur during or shortly after the administration of the drug and include symptoms such as fever, chills, nausea, rash, shortness of breath, and low blood pressure. IRRs are more common with the first infusion, but they can generally be managed with premedication (such as antihistamines and corticosteroids) or by slowing the infusion rate. Monoclonal antibodies can sometimes activate the immune system in unintended ways, leading to immune-related side effects. For example, rituximab can lead to the reactivation of latent infections, such as hepatitis B. Ipilimumab, pembrolizumab, and nivolumab can cause autoimmune reactions, where the immune system starts attacking healthy tissues. Monoclonal antibodies like trastuzumab can cause cardiotoxicity. Some monoclonal antibodies, such as rituximab, can cause blood-related side effects, including low white blood cell counts (neutropenia), low platelet counts (thrombocytopenia), and anemia. Nivolumab and pembrolizumab can cause liver inflammation and damage. Some monoclonal antibodies, particularly those targeting immune checkpoints, can interfere with normal endocrine function.

Antibody-drug conjugates (ADCs) are a class of cancer therapies that combine the targeting power of monoclonal antibodies with the potent cytotoxic effects of chemotherapy drugs. By linking a potent drug (often a chemotherapy agent or a cytotoxic substance) to a monoclonal antibody, ADCs can selectively deliver the drug directly to cancer cells, sparing healthy tissue and reducing systemic toxicity. This targeted approach has shown promise in treating cancers that are difficult to treat with conventional therapies. However, as with any treatment, ADCs have their own unique tradeoffs between efficacy and side effects. The effectiveness of ADCs depends on type of cancer, expression of the target antigen on cancer cells, potency of the cytotoxic drug, and the specific linker used to connect the antibody and the drug, among other factors. While ADCs are designed to be more targeted and less toxic than traditional chemotherapy, they still carry a risk of side effects, some of which are related to the chemotherapy drug being delivered to the cancer cells and others that arise from the targeting mechanism itself. The side effects can vary depending on the specific ADC and the tumor type, but common side effects including infusion-related reactions, hematologic toxicity, peripheral neuropathy, gastrointestinal dysfunctions, liver and pulmonary toxicity, cardiotoxicity, skin reactions, and tumor lysis syndrome.

Developing therapies that could improve the efficacy of known treatments while helping the body manage the side effects of cancer treatments is essential.

Isotope selective modulators is a class of drugs that are designed to repair cellular functions by selectively binding to target molecules inside a cell based upon the isotopic coordination with atoms of the target molecule. In oncology, isotope selective modulators combine their targeting power and potent cytotoxic effects with the mechanisms of action of the above cited therapies, aiming to help the immune system to recognize and fight cancer while reducing adverse effects by improving natural cellular functions.

Unlike the anticancer therapies cited above, which affect both cancerous and healthy cells, isotope selective modulators are designed to kill malignant cells while promoting healthy cellular functions in a dose-dependent manner. Their efficacy depends on several factors, including cancer type, atomic specifics of target proteins and enzymes, and isotopic signatures of the target tumor, among other factors. Isotope selective modulation therapies are designed to be inherently safe, aiming to have side effects ranging from none to mild. The first-in-humans, isotope selective modulator, KLS-1 (⁶⁴Znₑ-Asp), is in Phase 1 clinical trial for investigating its safety and side effects.

The development of organic compounds has traditionally relied on naturally occurring isotopes of chemical elements. The use of enriched stable isotopes in pharmaceuticals as part of organic molecules commenced with the discovery of deuterium by Harold Urey in 1932. For the first several decades, the focus was on understanding biological and chemical processes, leveraging the ability of stable isotopes like deuterium (Rita Maria Concetta Di Martino, 2023) and tritium to be detected through mass spectrometry and radioactivity measurements, respectively. Despite tritium being a radioactive isotope, its long half-life and the stable nature of deuterium allowed for safe handling and application in various biomedical areas. The concept of deuteration (substituting a hydrogen atom with deuterium atom) emerged as a significant advancement in drug discovery in the following decades. This innovative approach emerged with the development of deutetrabenazine (U.S. Pat. No. 8,524,733) approved by U.S. FDA in 2017 and deucravacitinib (WO 2018/183656) approved in 2022. Both deutetrabenazine and deucravacitinib use isotopically modified hydrogen ¹H. These breakthroughs marked the beginning of a new era in pharmaceutical design employing the isotopic modification of stable isotopes.

Recently, a number of works have demonstrated that isotopic composition of cancerous tissues and organs features distinct isotopic signatures. Breast carcinoma tumors were found to have a significantly lighter Zn isotopic composition than blood, serum and healthy breast tissue (Schilling et al., Front Med (Lausanne). 2022 Jan. 20; 8:746532. doi: 10.3389/fmed.2021.746532) Prostate cancer patients showed lower urinary δ⁶⁶Zn values compared to healthy controls. The δ⁶⁶Zn values for pancreatic adenocarcinoma, which makes ~90% of all pancreatic cancers, showed to be ranging between −0.33‰ to +0.15‰, with a median value of −0.15‰. Healthy controls tend to have higher δ66Zn values, ranging between −0.26‰ to 0.67‰, with a median value of +0.02‰. The δ⁶⁶Zn notation represents the relative difference in the ⁶⁶Zn/⁶⁴Zn ratio between a sample and a standard, typically expressed in parts per thousand (‰.) Hence, a decreased δ66Zn value refers to the increased ⁶⁴Zn fraction. The same is true for copper: an increased δ⁶⁵Cu value refers to the decreased ⁶³Cu fraction.

Further, certain patents and patent applications discuss the use of isotope selective modulators for therapeutic use. See, e.g., U.S. Pat. Nos. 9,861,659; 10,183,041, and 12,138,322, and U.S. patent application Ser. No. 18/780,726.

Combination therapies in oncology involve using more than one treatment modality to target cancer from different angles, with the goal of improving treatment efficacy, overcoming resistance mechanisms, and reducing the risk of relapse. Cancer cells can adapt and develop resistance to single therapies, so combining different types of treatments can enhance overall outcomes.

In one aspect, this disclosure provides a method of treating cancer comprising administering to a patient in need thereof a therapeutically effective amount of one or more anti-cancer agents selected from the group consisting of a chemotherapeutic agent, an immunotherapeutic agent, a kinase inhibitor, a monoclonal antibody, and an antibody-drug conjugate, and at least one isotope selective modulator inclusive of at least one isotope selected from the group consisting of ⁶³Cu, ⁶⁴Zn, ⁶⁶Zn, and ⁸⁵Rb, wherein said isotope is enriched to exceed 99% atomic mass.

In another aspect, this disclosure provides a method of treating cancer metastasis comprising administering to a patient in need thereof a therapeutically effective amount of one or more anti-cancer agents selected from the group consisting of a chemotherapeutic agent, an immunotherapeutic agent, a kinase inhibitor, a monoclonal antibody, and an antibody-drug conjugate, and at least one isotope selective modulator inclusive of at least one isotope selected from the group consisting of ⁶³Cu, ⁶⁴Zn, ⁶⁶Zn, and ⁸⁵Rb, wherein said isotope is enriched to exceed 99% atomic mass.

In some embodiments, the isotope selective modulator is administered prior to, concurrently with, or sequential to administering said one or more cancer therapies.

In some embodiments, the isotope selective modulator is administered intravenously. In some embodiments, the isotope selective modulator is administered intratumorally.

In some embodiments, the isotope selective modulator is administered in combination with at least one amino acid enriched to at least 90% L-enantiomer, a flavonoid, a carotenoid, or a polyphenol.

The cancer or cancer metastasis are ones meant to be treated by the one or more anti-cancer agents/cancer therapies.

In some embodiments, the isotope selective modulator and the one or more anti-cancer agents/cancer therapies have a synergistic effect on treating the cancer or cancer metastasis of the patient. In other embodiments, the isotope selective modulator and the one or more anti-cancer agents/cancer therapies have an additive effect on treating the cancer or cancer metastasis of the patient. In some embodiments, the isotope selective modulator and the one or more anti-cancer agents/cancer therapies do not have any negative effect of the other's anti-cancer action.

In one aspect, a method is provided for treating cancer including administering a chemotherapeutic agent and administering an isotope selective modulator. Administering chemotherapy can include an intravenous injection or infusion of alkylating agents, antimetabolites, or topoisomerase inhibitors, or a combination of these methods, or other chemotherapy treatment methods. In some embodiments, administering an isotope selective modulator includes an intravenous injection or infusion of ⁶⁴Znₑ(Asp).

In one embodiment, the chemotherapy treatment is administered prior to administering an isotope selective modulator to the patient. In another embodiment, the chemotherapy treatment is administered concurrently with administering an isotope selective modulator to the patient. In yet another embodiment, the chemotherapy treatment is administered after administering an isotope selective modulator to the patient.

The isotope selective modulator is administered at any suitable dose, including a therapeutically effective or a prophylactic dose.

In certain embodiments, the isotope selective modulator is a ⁶⁴Znₑ (Asp) chelate inclusive of aspartic acid. In certain embodiments, the isotope selective modulator is a ⁶⁴Znₑ chelated with glutamic acid. In certain embodiments, the isotope selective modulator is a ⁶⁴Znₑ enriched to 99.2% and chelated with glutamate. In certain embodiments, the isotope selective modulator is a ⁶³Cuₑ enriched to 96% and chelated with tyrosine. In certain embodiments, the isotope selective modulator is ⁸⁵Ruₑ enriched to 99% and chelated with proline and added alanine.

In certain embodiments, ⁶⁴Znₑ(Asp), or zinc aspartate, has a chemical formula-(C₄H₅O₄N)₂⁶⁴Znₑ, with 2 aspartic acid molecules. In another embodiment, ⁶⁴Znₑ(Asp), or zinc aspartate has a chemical formula

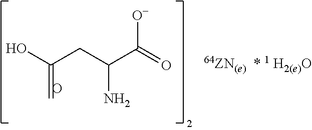

In another aspect, a method is provided for treating cancer metastases comprising administering a chemotherapy and administering an isotope selective modulator. In one embodiment, the isotope selective modulator may be administered intravenously. In another embodiment, the isotope selective modulator may be administered intratumorally.

In certain other embodiments, the amino acid chelating an isotope selective modulator may be enriched for the L-enantiomer for at least 90% L-enantiomer.

As used herein, unless otherwise indicated, X % ⁶⁴Zn means that, out of 100 zinc atoms, X is ⁶⁴Zn. For example, in an isotope selective modulator that includes zinc that is 99% ⁶⁴Zn, 99% of zinc atoms are ⁶⁴Zn. Likewise, X % ⁶³Cu means that, out of 100 copper atoms, X is ⁶³Cu. For example, in an isotope selective modulator that includes copper that is 90% ⁶³Cu, 90% of copper atoms are ⁶³Cu. The same is true for ⁸⁵Rb-X % ⁸⁵Rb means that, out of 100 rubidium atoms, X is ⁸⁵Rb.

Unless otherwise indicated, the term “⁶⁴Zn.” is used herein as shorthand for “⁶⁴Zn-enriched zinc”. The term “⁶³Cuₑ” is used herein as shorthand for “⁶³Cu-enriched copper”. The term “⁸⁵Rb.” is used herein as shorthand for “⁸⁵Rb-enriched rubidium.”

The mode of administration of the disclosed active ingredients can depend on the conditions or disease to be targeted or treated. The selection of the specific route of administration can be selected or adjusted by the clinician according to methods known to the clinician to obtain the desired clinical response. The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

In certain embodiments, enriched ⁶³Cu, ⁶⁴Zn, ⁶⁶Zn, or ⁸⁵Rb isotopes are active ingredients chelated by amino acids, as specified. Utilizing the teachings provided in the present disclosure, an effective prophylactic or therapeutic treatment regimen may be planned that is effective to treat the clinical symptoms demonstrated by the particular patient without causing substantial toxicity. Such planning should involve careful choice of active chemotherapy, immunotherapy, kinase inhibitor, monoclonal antibody, or antibody-drug conjugate therapy by considering their potency, patient condition, severity of anticipated adverse effects, preferred mode of administration, and the toxicity profile of the selected agent, and clinical pathways subject to modulation by an enriched isotope comprised in an isotope selective modulator.

### EXAMPLES

For this invention to be better understood, the following examples are set forth. These examples are for purposes of illustration only and are not be construed as limiting the scope of the invention in any manner.

### Example 1. Combination Therapy Using an Anti-Cancer Therapy and an Isotope Selective Modulator In Vitro Experiments on Melanoma Cells

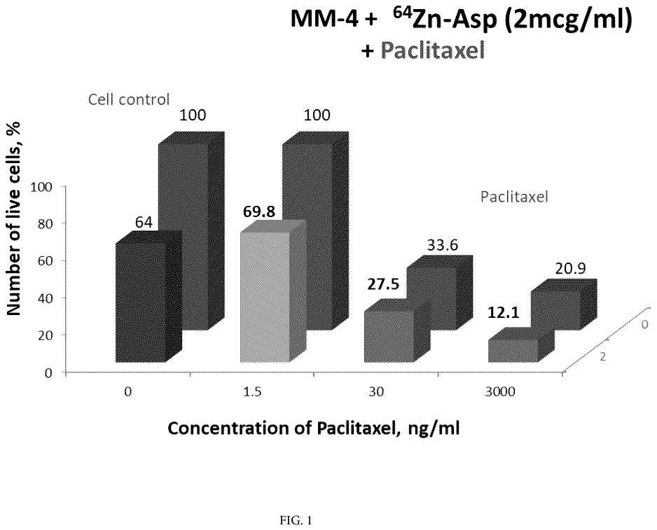

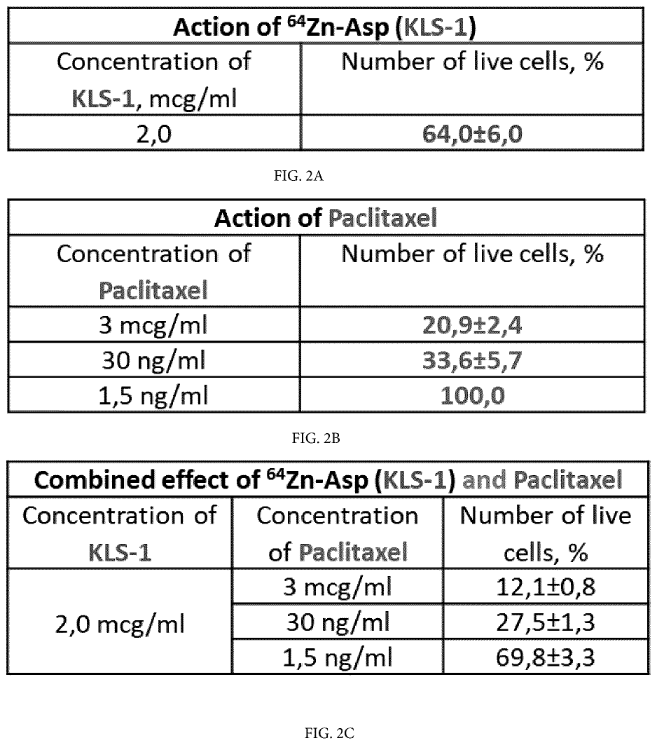

Paclitaxel and ⁶⁴Znₑ-Aspartate act as independent cytotoxic agents to melanoma cells in vitro experiments, rendering no cross-negating effect on each other cytotoxic action to melanoma cells (MM-4 cells). FIG. 1 and FIG. 2. In this embodiment, the isotope selective modulator was administered 24 hours prior to Paclitaxel. It is essential that co-treatments do not diminish each other's intended action.

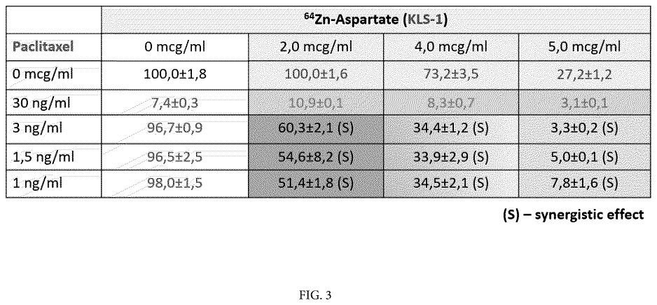

Paclitaxel and ⁶⁴Znₑ-Aspartate act as independent cytotoxic agents, rendering pharmacological synergism exhibiting dual anti-proliferative/cytotoxic impact on melanoma cells. FIG. 3. The isotope selective modulator was administered 24 hours after Paclitaxel. It is essential that co-treatments enhance each other's intended action. Pharmacological synergism makes it possible to obtain a better therapeutic effect of drugs in lower doses.

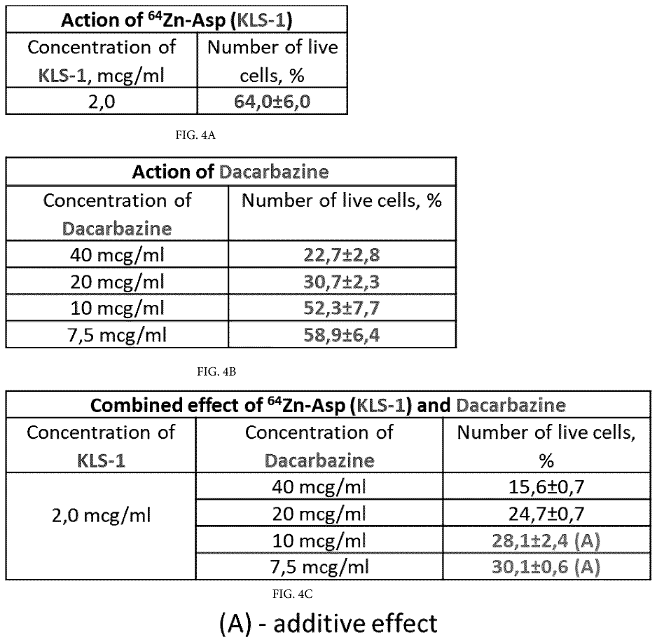

Dacarbazine and ⁶⁴Znₑ-Aspartate act as independent cytotoxic agents, rendering an additive effect on each other cytotoxic action to melanoma cells. FIG. 4. The isotope selective modulator was administered 24 hours prior to Dacarbazine. It is essential that co-treatments enhance each other's intended action.

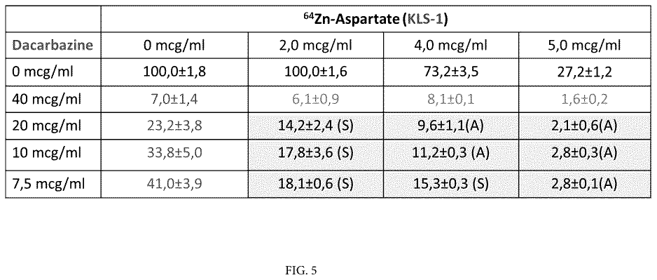

Dacarbazine and ⁶⁴Znₑ-Aspartate act as independent cytotoxic agents, rendering both an additive effect and a synergistic effect on each other cytotoxic action to melanoma cells. FIG. 5. The isotope selective modulator was administered 24 hours after Dacarbazine. It is essential that co-treatments enhance each other's intended action.

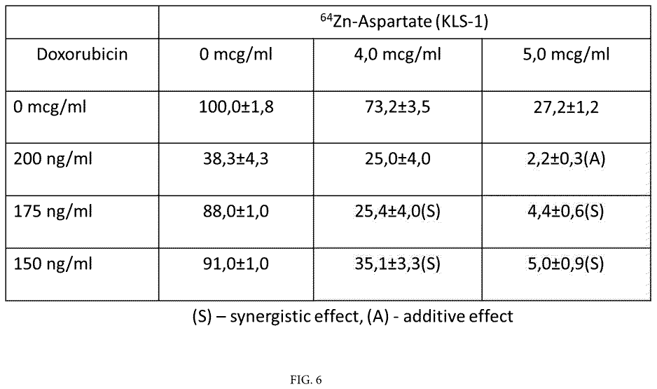

Doxorubicin and ⁶⁴Znₑ-Aspartate act as independent cytotoxic agents, rendering both an additive effect and a synergistic effect on each other cytotoxic action to melanoma cells. FIG. 6. The isotope selective modulator was administered 24 hours after Doxorubicin. It is essential that co-treatments enhance each other's intended action.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A method of treating a primary melanoma cancer with a combination therapy comprising administering to a patient in need thereof:
(i) a therapeutically effective amount of one or more anticancer agents selected from the group consisting of a chemotherapeutic agent, an immunotherapeutic agent, a kinase inhibitor, a monoclonal antibody, and an antibody-drug conjugate, and
(ii) a therapeutically effective amount of at least one isotope selective modulator inclusive of at least one isotope selected from the group consisting of ⁶³Cu, ⁶⁴Zn, ⁶⁶Zn, and ⁸⁵Rb, wherein said isotope is enriched to exceed 99% atomic mass;
wherein said anti-cancer agent(s) are known to be effective against primary melanoma cancer.

2. The method of claim 1, wherein said isotope selective modulator is administered prior to, concurrently with, or sequentially to administering said one or more cancer therapies.

3. The method of claim 1, wherein said isotope selective modulator is administered intravenously.

4. The method of claim 1, wherein said isotope selective modulator is administered intratumorally.

5. The method of claim 1, wherein said isotope selective modulator is optionally administered in combination with at least one amino acid enriched to at least 90% L-enantiomer, a flavonoid, a carotenoid, or a polyphenol.

6. A method of treating a metastatic melanoma cancer with a combination therapy comprising administering to a patient in need thereof:
(i) a therapeutically effective amount of one or more anti-cancer agents selected from the group consisting of a chemotherapeutic agent, an immunotherapeutic agent, a kinase inhibitor, a monoclonal antibody, and an antibody-drug conjugate, and
(ii) a therapeutically effective amount of at least one isotope selective modulator inclusive of at least one isotope selected from the group consisting of ⁶³Cu, ⁶⁴Zn, ⁶⁶Zn, and ⁸⁵Rb, wherein said isotope is enriched to exceed 99% atomic mass;
wherein said anti-cancer agent(s) are known to be effective against said metastatic melanoma cancer.

7. The method of claim 6, wherein said isotope selective modulator is administered prior to, concurrently with, or sequential to administering said one or more cancer therapies.

8. The method of claim 6, wherein said isotope selective modulator is administered intravenously.

9. The method of claim 6, wherein said isotope selective modulator is administered intratumorally.

10. The method of claim 6, wherein said isotope selective modulator is optionally administered in combination with at least one amino acid enriched to at least 90% L-enantiomer, a flavonoid, a carotenoid, or a polyphenol.
